(19) United States
(12) Patent Application Publication
(10) Pub. No.: US 2025/0024860 A1
(43) Pub. Date: Jan. 23, 2025

(54) COMPOSITIONS AND METHODS FOR TREATING OR PREVENTING MYOPATHY IN POULTRY

(71) Applicants: Nutrition21, LLC, Saddle Brook, NJ (US); Kent Corporation, Muscatine, IA (US)

(72) Inventors: William Hammock, Melbourne, FL (US); Douglas A. Stetzer, Superior, WI (US); Xiaowen Guo, Muscatine, IA (US)

(21) Appl. No.: 18/709,183

(22) PCT Filed: Nov. 11, 2022
(86) PCT No.: PCT/US22/49703

§ 371 Date: May 10, 2024

Related U.S. Application Data
(60) Provisional application No. 63/278,987, filed on Nov. 12, 2021.

(51) Int. Cl. A23K 20/142; A23K 10/30; A23K 20/195; A23K 20/28; A23K 50/75; A61K 31/198; A61P 21/00

(52) U.S. Cl. CPC A23K 20/142; A23K 10/30; A23K 20/195; A23K 20/28; A23K 50/75; A61K 31/198; A61P 21/00

(57) ABSTRACT

Provided are methods of feeding a bird or feeding a population of birds by administering arginine silicate inositol. Also provided are methods of treating or preventing a myopathy in poultry. Also provided are methods for increasing poultry weight and improving poultry breast fillet quality. The methods include administering arginine silicate inositol or a composition comprising arginine silicate inositol to a bird or a population of birds. Also provided are compositions comprising arginine silicate inositol and at least one carrier.

### RELATED APPLICATIONS

This application claims the benefit of U.S. Provisional Application No. 63/278,987, filed Nov. 12, 2021, the contents of which are fully incorporated by reference herein.

### BACKGROUND

Poultry is the largest source of animal protein in the human diet. To meet the growing demand for chicken, by far the most consumed poultry meat, many producers raise broilers selectively bred to maximize yield and minimize time from birth to slaughter weight. Additionally, the broilers are often provided a feed designed to maximize their edible weight per weight of feed consumed. Although these interventions have led to an increase in broiler yield and an improvement in feed efficiency, they have been associated with a rise in the incidence of myopathies in the broilers. White striping and woody breast, two myopathies that were almost nonexistent a couple decades ago, are now commonly detected in commercial chicken breasts. White striping presents as white striations of fat deposits parallel to muscle fibers on breast meat while fillets with woody breast exhibit discoloration and a rigid, rubbery, and swollen texture. These myopathies diminish the quality and appearance of breast meat, leading to a reduction in consumer satisfaction or outright consumer rejection. As such, it would be desirable to provide compositions and methods for inhibiting myopathies such as white striping and woody breast without sacrificing yield or feed efficiency.

### SUMMARY

In certain aspects, provided herein is a method of feeding a bird, comprising feeding a composition comprising arginine silicate inositol to the bird.

In further aspects, provided herein is a method of feeding a population of birds, comprising feeding a composition comprising arginine silicate inositol to the population of birds.

In further aspects, provided herein is a method for treating, inhibiting, or preventing a myopathy comprising administering arginine silicate inositol to a bird.

In further aspects, provided herein is a method for treating, inhibiting, or preventing a myopathy comprising administering arginine silicate inositol to a population of birds.

In further aspects, provided herein is a method for improving poultry breast fillet quality comprising administering arginine silicate inositol to a bird.

In further aspects, provided herein is a method for improving poultry breast fillet quality comprising administering arginine silicate inositol to a population of birds.

In further aspects, provided herein is a method for increasing poultry weight comprising administering arginine silicate inositol to a bird.

In further aspects, provided herein is a method for increasing poultry weight comprising administering arginine silicate inositol to a population of birds.

In further aspects, provided herein is a composition comprising from about 0.005 wt. % to about 0.5 wt. % arginine silicate inositol and at least one carrier. The composition may be a liquid or solid composition, particularly a bird feed composition.

### BRIEF DESCRIPTION OF THE DRAWINGS

FIG. 1 depicts the feed intake results of Example 1.

FIG. 2 depicts the body weight gain results of Example 1.

FIG. 3 depicts the body weight results of Example 1.

FIG. 4 depicts the feed conversion ratio results of Example 1.

FIG. 5 depicts the breast width results of Example 1.

FIG. 6 depicts the fillet weight results of Example 1.

FIG. 7 depicts the compression force results of Example 1.

FIG. 8 depicts the water holding capacity results of Example 1.

FIG. 9A-9D depict the day 42 breast fillet palpation woody breast score results of Example 1. FIG. 9A depicts the Diet 1 results. FIG. 9B depicts the Diet 2 results. FIG. 9C depicts the Diet 3 results. FIG. 9D depicts the Diet 4 results.

FIG. 10A-10D depict the day 49 breast fillet palpation woody breast score results of Example 1. FIG. 10A depicts the Diet 1 results. FIG. 10B depicts the Diet 2 results. FIG. 10C depicts the Diet 3 results. FIG. 10D depicts the Diet 4 results.

### DETAILED DESCRIPTION OF THE INVENTION

In certain aspects, provided herein are compositions and methods for treating, inhibiting, or preventing myopathy in poultry. The methods described herein comprise administering arginine silicate inositol (ASI) to a bird. The methods may treat, inhibit, or prevent myopathies such as white striping and woody breast in the bird without reducing yield or feed efficiency. As a consequence, breast fillets excised from birds that are administered ASI may exhibit superior tenderness, taste, appearance, and texture compared with breast fillets excised from birds that are not administered ASI. Without intending to be bound by theory, it is believed that birds that grow muscle too quickly have a reduced capillary to muscle fiber ratio. The resulting insufficient capillary network may cause the muscle fibers to degenerate and disorganize. ASI may act as a vasodilator, which improves circulation and thus reduces muscle degeneration. Besides treating, inhibiting, or preventing myopathy in the bird, the methods may increase the weight of the bird and improve poultry breast fillet quality.

### Methods

In certain aspects, provided herein is a method of feeding a bird, comprising feeding a composition comprising ASI to the bird.

In further aspects, provided herein is a method of feeding a population of birds, comprising feeding a composition comprising ASI to the population of birds.

In further aspects, provided herein is a method for treating, inhibiting, or preventing a myopathy comprising administering ASI to a bird.

In some embodiments, the ASI is prepared using the processes described in U.S. Pat. Nos. 5,707,970, 6,803,456, and 11,103,000, each of which are incorporated by reference herein in their entirety.

In certain embodiments, the ASI has a molar ratio of arginine to silicate of from about 0.5:1 to about 2:1, from about 0.75:1 to about 1.25:1, from about 0.8:1 to about 1.2:1, or about 1:1. In some embodiments, the ASI has a molar ratio of arginine to inositol of from about 1:1 to about 4:1, from about 1.25:1 to about 3:1, from about 1.5:1 to about 3:1, or about 2:1. According to one or more embodiments, the ASI has a molar ratio of arginine to silicate to inositol of about 3:3:1 or about 2:2:1. In certain embodiments, the ASI has a molar ratio of arginine to silicate to inositol of about 2:2:1. In some embodiments, the ASI has a molar ratio of arginine to silicate to inositol that is suitable for consumption by a bird. In certain embodiments, the ASI is administered to the bird orally (by ingestion), intravenously, arterially, intradermally, intramuscularly, intraperitoneally, subcutaneously, sublingually, intranasally, or transdermally. In some embodiments, the ASI is administered orally. In certain embodiments, the ASI is administered in a liquid. In some embodiments, the ASI is administered in water. According to one or more embodiments, the ASI is administered topically under a wing of the bird. In certain embodiments, the ASI is administered parenterally. In some embodiments, the ASI is administered under a wing of the bird or in the nostril of the bird.

In some embodiments, the ASI is formulated for extended release, controlled release, or a combination thereof.

In some embodiments, the bird or population of birds is in need of treatment.

In certain embodiments, the myopathy is white striping, woody breast, spaghetti meat, or deep pectoral myopathy. In some embodiments, the myopathy is white striping or woody breast. In certain embodiments, the myopathy is woody breast.

In some embodiments, the bird is a turkey, a quail, a goose, a duck, a pigeon, a guinea fowl, or a chicken. In certain embodiments, the bird is a chicken or a turkey. According to one or more embodiments, the bird is a chicken. In some embodiments, the chicken is a Cornish Cross, White Rock, Big Red Broiler, Breese, Turken, Kosher King, Dark Cornish, Ginger Broiler, Jersey Giant, Orpington, Delaware, Buckeye, Chantecler, Dorking, New Hampshire Red, Rhode Island Red, or a crossbreed thereof. In some embodiments, the chicken is a broiler. In certain embodiments, the broiler is a Ross® 708, a Ross® 308, or a Cobb500™. According to one or more embodiments, the broiler is a Ross® 708.

In some embodiments, the ASI is administered as a composition comprising ASI and at least one carrier.

In certain embodiments, the composition is a liquid composition.

In some embodiments, the carrier is water or an oil. In certain embodiments, the oil is peanut oil, olive oil, a liquid paraffin, palm oil, sunflower oil, soy oil, linseed oil, or a combination thereof.

In some embodiments, the liquid composition is an emulsion, a suspension, or a solution.

According to one or more embodiments, the ASI is suspended in the carrier. In some embodiments, the ASI is dissolved in the carrier.

In some embodiments, the liquid composition further comprises a sweetener, a flavoring agent, a coloring agent, a preservative, a solubilizer, a wetting agent, a stabilizer, an antioxidant, a diluent, or a combination thereof.

In certain embodiments, the composition is a solid composition. According to one or more embodiments, the carrier is an animal feed. In some embodiments, the solid composition is formulated as a mash, a crumble, a scratch grain, or pellets. In some embodiments, the composition is a solid composition and the carrier is an animal feed.

In certain embodiments, the animal feed comprises a cereal grain, an oilseed meal, or a combination thereof. In some embodiments, the animal feed comprises corn, wheat, soybean, sorghum, barley, rye, fishmeal, peas, flax meal, sunflower meal, lupins, canola meal, or a combination thereof. In some embodiments, the animal feed comprises a cereal grain selected from corn, soybean, wheat, or a combination thereof. According to one or more embodiments, the animal feed comprises a cereal grain at a concentration of from about 50 wt. % to about 90 wt. %, from about 55 wt. % to about 85 wt. %, from about 55 wt. % to about 80 wt. %, from about 55 wt. % to about 75 wt. %, or from about 60 wt. % to about 70 wt. %. In some embodiments, the animal feed comprises an oilseed meal at a concentration of from about 5 wt. % to about 40 wt. %, from about 10 wt. % to about 35 wt. %, from about 10 wt. % to about 30 wt. %, from about 15 wt. % to about 25 wt. %, or from about 18 wt. % to about 22 wt. %. In certain embodiments, the animal feed comprises protein at a concentration of from about 10 wt. % to about 50 wt. %, from about 12 wt. % to about 50 wt. %, from about 12 wt. % to about 40 wt. %, from about 12 wt. % to about 35 wt. %, from about 12 wt. % to about 30 wt. %, from about 12 wt. % to about 25 wt. %, from about 12 wt. % to about 22 wt. %, from about 12 wt. % to about 20 wt. %, or from about 14 wt. % to about 18 wt. %.

In certain embodiments, the animal feed comprises a fat or oil. In some embodiments, the animal feed comprises pig fat, beef fat, linseed oil, soy oil, sunflower oil, palm oil, or a combination thereof.

In certain embodiments, the animal feed comprises a coccidiostat, an antibiotic, or a combination thereof. In some embodiments, the animal feed comprises monensin sodium, lasalocid, amprolium, toltrazuril, salinomycin, or a combination thereof. According to one or more embodiments, the animal feed comprises penicillin, bacitracin, chlortetracycline, oxytetracycline, or a combination thereof.

In certain embodiments, the animal feed comprises prebiotics and probiotics.

In certain embodiments, the animal feed comprises an added mineral. In some embodiments, the animal feed comprises added copper, iodide, iron, manganese, selenium, zinc, or a combination thereof.

In certain embodiments, the animal feed comprises an added vitamin. In some embodiments, the animal feed comprises added vitamin A, vitamin D₃, vitamin E, vitamin K, thiamin, riboflavin, niacin, pantothenic acid, pyridoxine, biotin, folic acid, vitamin B₁₂, or a combination thereof.

In certain embodiments, the animal feed comprises an added mineral. In some embodiments, the animal feed comprises added calcium, phosphorous, magnesium, sodium, chloride, potassium, or a combination thereof. In some embodiments, the animal feed comprises added monocalcium phosphate, limestone, or a combination thereof.

In certain embodiments, the animal feed comprises an added digestible amino acid other than arginine. In some embodiments, the animal feed comprises added lysine, methionine, cysteine, threonine, valine, isoleucine, tryptophan, leucine, or a combination thereof.

In certain embodiments, the animal feed comprises a sweetener, a flavoring agent, a coloring agent, a preservative, a stabilizer, an antioxidant, or a coating agent.

In some embodiments, the solid composition is adapted to suit an age of the bird because the bird has different nutritional needs at different ages. In certain embodiments, the solid composition is adapted for a starter period, a grower period, a finisher 1 period, or a finisher 2 period. In some embodiments, the starter period is defined herein as an age of from about 0 days to about 14 days. In certain embodiments, the grower period is defined herein as an age of about 14 days to about 28 days. According to one or more embodiments, the finisher 1 period is defined herein as an age of about 28 days to about 42 days. In certain embodiments, the finisher 2 period is defined herein as an age of about 42 days to about 49 days.

In some embodiments, the solid composition is adapted to have a low feed conversion ratio (FCR). The FCR as defined herein is the weight of the solid composition intake divided by the weight gain of the bird. In certain embodiments, the solid composition is adapted to provide an FCR of from about 0.8 to about 1.0 during the starter period. In some embodiments, the solid composition is adapted to provide an FCR of from about 1.0 to about 1.47 during the grower period. According to one or more embodiments, the solid composition is adapted to provide an FCR of from about 1.4 to about 1.55 during the finisher 1 period. In certain embodiments, the solid composition is adapted to provide an FCR of from about 2.2 to about 2.3 during the finisher 2 period.

In certain embodiments, the bird is administered from 0.1 kg to 10 kg, 0.2 kg to 0.5 kg, from 1.5 kg to 2.0 kg, from 2.4 kg to 2.8 kg, or from 1.3 kg to 1.5 kg of the solid composition.

In some embodiments, the composition comprises ASI at a concentration of from about 0.001 wt. % to about 10 wt. %, from about 0.5 wt. % to about 10 wt. %, from about 0.001 wt. % to about 5 wt. %, from about 0.001 wt. % to about 2 wt. %, from about 0.001 wt. % to about 1 wt. %, from about 0.001 wt. % to about 0.5 wt. %, from about 0.002 wt. % to about 0.5 wt. %, from about 0.005 wt. % to about 0.5 wt. %, from about 0.01 wt. % to about 0.2 wt. %, from about 0.02 wt. % to about 0.03 wt. %, from about 0.03 wt. % to about 0.08 wt. %, from about 0.08 wt. % to about 0.12 wt. %, from about 0.12 wt. % to about 0.16 wt. %, about 0.025 wt. %, about 0.05 wt. %, about 0.10 wt. %, or about 0.15 wt. %.

In certain embodiments, the composition comprises ASI at a concentration of from about 10 wt. % to about 99.9 wt. %, from about 10 wt. % to about 95 wt. %, from about 10 wt. % to about 90 wt. %, from about 10 wt. % to about 80 wt. %, from about 10 wt. % to about 70 wt. %, from about 10 wt. % to about 60 wt. %, from about 10 wt. % to about 50 wt. %, from about 10 wt. % to about 40 wt. %, from about 10 wt. % to about 30 wt. %, from about 10 wt. % to about 20 wt. %, from about 20 wt. % to about 99.9 wt. %, from about 20 wt. % to about 95 wt. %, from about 20 wt. % to about 90 wt. %, from about 20 wt. % to about 80 wt. %, from about 20 wt. % to about 70 wt. %, from about 20 wt. % to about 60 wt. %, from about 20 wt. % to about 50 wt. %, from about 20 wt. % to about 40 wt. %, from about 20 wt. % to about 30 wt. %, from about 30 wt. % to about 99.9 wt. %, from about 30 wt. % to about 95 wt. %, from about 30 wt. % to about 90 wt. %, from about 30 wt. % to about 80 wt. %, from about 30 wt. % to about 70 wt. %, from about 30 wt. % to about 60 wt. %, from about 30 wt. % to about 50 wt. %, from about 30 wt. % to about 40 wt. %, from about 40 wt. % to about 99.9 wt. %, from about 40 wt. % to about 95 wt. %, from about 40 wt. % to about 90 wt. %, from about 40 wt. % to about 80 wt. %, from about 40 wt. % to about 70 wt. %, from about 40 wt. % to about 60 wt. %, from about 40 wt. % to about 50 wt. %, from about 50 wt. % to about 99.9 wt. %, from about 50 wt. % to about 95 wt. %, from about 50 wt. % to about 90 wt. %, from about 50 wt. % to about 80 wt. %, from about 50 wt. % to about 70 wt. %, from about 50 wt. % to about 60 wt. %, from about 60 wt. % to about 99.9 wt. %, from about 60 wt. % to about 95 wt. %, from about 60 wt. % to about 90 wt. %, from about 60 wt. % to about 80 wt. %, from about 60 wt. % to about 70 wt. %, from about 70 wt. % to about 99.9 wt. %, from about 70 wt. % to about 95 wt. %, from about 70 wt. % to about 90 wt. %, from about 70 wt. % to about 80 wt. %, from about 80 wt. % to about 99.9 wt. %, from about 80 wt. % to about 95 wt. %, from about 80 wt. % to about 90 wt. %, from about 90 wt. % to about 99.9 wt. %, from about 90 wt. % to about 95 wt. %, from about 95 wt. % to about 99.9 wt. %, from about 98 wt. % to about 99.9 wt. %, or from about 99 wt. % to about 99.9 wt. %.

In certain embodiments, the ASI is administered over from 1 day to 55 days, from 1 day to 49 days, from 1 day to 7 days, from 1 day to 14 days, from 1 day to 28 days, from 1 day to 35 days, from 1 day to 42 days, from 5 days to 7 days, from 5 days to 55 days, from 5 days to 49 days, from 5 days to 14 days, from 5 days to 28 days, from 5 days to 35 days, from 5 days to 42 days, from 7 days to 55 days, from 7 days to 49 days, from 7 days to 14 days, from 7 days to 28 days, from 7 days to 35 days, from 7 days to 42 days, from 10 days to 55 days, from 10 days to 49 days, from 10 days to 14 days, from 10 days to 28 days, from 10 days to 35 days, from 10 days to 42 days, from 14 days to 55 days, from 10 days to 49 days, from 14 days to 28 days, from 14 days to 35 days, from 14 days to 42 days, from 20 days to 55 days, from 20 days to 49 days, from 20 days to 28 days, from 20 days to 35 days, from 20 days to 42 days, from 28 days to 55 days, from 28 days to 49 days, from 28 days to 35 days, from 28 days to 42 days, from 35 days to 55 days, from 35 days to 49 days, or from 35 days to 42 days.

In certain embodiments, the method comprises slaughtering the bird at an age of from 38 days to 55 days, from 40 days to 50 days, from 40 days to 43 days, from 41 to 43 days, from 43 days to 50 days, from 43 days to 47 days, from 45 days to 49 days, from 48 days to 50 days, from 45 days to 48 days, from 47 days to 50 days, from 48 days to 50 days, 42 days, 45 days, 47 days, or 49 days.

In some embodiments, the bird weighs from about 1.5 kg to about 2.5 kg, from about 3.0 kg to about 3.5 kg, from about 3.3 kg to about 3.4 kg, from about 3.7 kg to about 4.0 kg, or from about 3.88 kg to about 4.00 kg when slaughtered.

In certain embodiments, the composition is formulated in a tablet, a hard capsule, or a soft capsule. According to one or more embodiments, the carrier is a pharmaceutically acceptable excipient. In some embodiments, the pharmaceutically acceptable excipient is calcium carbonate, sodium carbonate, lactose, calcium phosphate, sodium phosphate, corn starch, alginic acid, starch, gelatin, acacia, magnesium stearate, stearic acid, talc, or a combination thereof. In some embodiments, the tablet, the hard capsule, or the soft capsule is uncoated or coated. In some embodiments, the tablet, the hard capsule, or the soft capsule an enteric coating. According to one or more embodiments, the tablet, the hard capsule, or the soft capsule comprises glyceryl monostearate or glyceryl distearate.

Besides treating, inhibiting, or preventing a myopathy in poultry, the methods according to embodiments described herein may increase the bird's feed intake, increase the bird's weight, improve the quality of breast fillets excised from the bird, increase the bird's feed conversion ratio, or increase the yield of the bird.

In certain aspects, provided herein is a method for improving poultry breast fillet quality comprising administering ASI to a bird.

In further aspects, provided herein is a method for improving poultry breast fillet quality comprising administering ASI to a population of birds.

In some embodiments, the breast fillet quality is improved relative to a population of birds to which the ASI is not administered.

In further aspects, provided herein is a method for increasing poultry weight comprising administering ASI to a bird.

In further aspects, provided herein is a method for increasing poultry weight comprising administering ASI to a population of birds.

In some embodiments, the average weight of the population of birds is increased relative to a population of birds to which the ASI is not administered.

### Compositions

In certain aspects, provided herein is a solid composition comprising from about 0.005 wt. % to about 0.5 wt. % ASI and at least one carrier.

According to one or more embodiments, the carrier is an animal feed. In some embodiments, the solid composition is formulated as a mash, a crumble, a scratch grain, or pellets.

In certain embodiments, the animal feed comprises a cereal grain, an oilseed meal, or a combination thereof. In some embodiments, the animal feed comprises corn, wheat, soybean, sorghum, barley, rye, fishmeal, peas, flax meal, sunflower meal, lupins, canola meal, or a combination thereof. In some embodiments, the animal feed comprises a cereal grain selected from corn, soybean, wheat, or a combination thereof. According to one or more embodiments, the animal feed comprises a cereal grain at a concentration of from about 50 wt. % to about 90 wt. %, from about 55 wt. % to about 85 wt. %, from about 55 wt. % to about 80 wt. %, from about 55 wt. % to about 75 wt. %, or from about 60 wt. % to about 70 wt. %. In some embodiments, the animal feed comprises an oilseed meal at a concentration of from about 5 wt. % to about 40 wt. %, from about 10 wt. % to about 35 wt. %, from about 10 wt. % to about 30 wt. %, from about 15 wt. % to about 25 wt. %, or from about 18 wt. % to about 22 wt. %. In certain embodiments, the animal feed comprises protein at a concentration of from about 10 wt. % to about 50 wt. %, from about 12 wt. % to about 50 wt. %, from about 12 wt. % to about 40 wt. %, from about 12 wt. % to about 35 wt. %, from about 12 wt. % to about 30 wt. %, from about 12 wt. % to about 25 wt. %, from about 12 wt. % to about 22 wt. %, from about 12 wt. % to about 20 wt. %, or from about 14 wt. % to about 18 wt. %.

In certain embodiments, the animal feed comprises a fat or oil. In some embodiments, the animal feed comprises pig fat, beef fat, linseed oil, soy oil, sunflower oil, palm oil, or a combination thereof.

In certain embodiments, the animal feed comprises a coccidiostat, an antibiotic, or a combination thereof. In some embodiments, the animal feed comprises monensin sodium, lasalocid, amprolium, toltrazuril, salinomycin, or a combination thereof. According to one or more embodiments, the animal feed comprises penicillin, bacitracin, chlortetracycline, oxytetracycline, or a combination thereof.

In certain embodiments, the animal feed comprises prebiotics and probiotics.

In certain embodiments, the animal feed comprises an added mineral. In some embodiments, the animal feed comprises added copper, iodide, iron, manganese, selenium, zinc, or a combination thereof.

In certain embodiments, the animal feed comprises an added vitamin. In some embodiments, the animal feed comprises added vitamin A, vitamin D₃, vitamin E, vitamin K, thiamin, riboflavin, niacin, pantothenic acid, pyridoxine, biotin, folic acid, vitamin B₁₂, or a combination thereof.

In certain embodiments, the animal feed comprises an added mineral. In some embodiments, the animal feed comprises added calcium, phosphorous, magnesium, sodium, chloride, potassium, or a combination thereof. In some embodiments, the animal feed comprises added monocalcium phosphate, limestone, or a combination thereof.

In certain embodiments, the animal feed comprises an added digestible amino acid other than arginine. In some embodiments, the animal feed comprises added lysine, methionine, cysteine, threonine, valine, isoleucine, tryptophan, leucine, or a combination thereof.

In certain embodiments, the animal feed comprises a sweetener, a flavoring agent, a coloring agent, a preservative, a stabilizer, an antioxidant, or a coating agent.

In some embodiments, the solid composition is adapted to suit an age of the bird because the bird has different nutritional needs at different ages. In certain embodiments, the solid composition is adapted for a starter period, a grower period, a finisher 1 period, or a finisher 2 period. In some embodiments, the starter period is defined herein as an age of from about 0 days to about 14 days. In certain embodiments, the grower period is defined herein as an age of about 14 days to about 28 days. According to one or more embodiments, the finisher 1 period is defined herein as an age of about 28 days to about 42 days. In certain embodiments, the finisher 2 period is defined herein as an age of about 42 days to about 49 days.

In some embodiments, the solid composition is adapted to provide a certain average feed intake per day per bird to a population of birds. In some embodiments, the solid composition is adapted to provide an average feed intake per day per bird during the starter period of about 25 g to about 35 g, about 28 g to about 31 g, about 29 g to about 31 g, or about 30 g. In some embodiments, the solid composition is adapted to provide an average feed intake per day per bird during the grower period of about 115 g to about 122 g, about 117 g to about 120 g, or about 120 g. In some embodiments, the solid composition is adapted to provide an average feed intake per day per bird during the finisher 1 period of about 170 g to about 200 g, about 184 g to about 190 g, about 186 g to about 188 g, or about 190 g. In some embodiments, the solid composition is adapted to provide an average feed intake per day per bird during the finisher 2 period of about 180 g to about 220 g, about 197 g to about 205 g, or about 200 g.

In some embodiments, the solid composition is adapted to have a low feed conversion ratio (FCR). The FCR as defined herein is the weight of the solid composition intake divided by the weight gain of the bird. In certain embodiments, the solid composition is adapted to provide an FCR of from about 0.8 to about 1.0 during the starter period. In some embodiments, the solid composition is adapted to provide an FCR of from about 1.0 to about 1.47 during the grower period. According to one or more embodiments, the solid composition is adapted to provide an FCR of from about 1.4 to about 1.55 during the finisher 1 period. In certain embodiments, the solid composition is adapted to provide an FCR of from about 2.2 to about 2.3 during the finisher 2 period.

In some embodiments, the solid composition comprises ASI at a concentration of from about 0.001 wt. % to about 10 wt. %, from about 0.5 wt. % to about 10 wt. %, from about 0.001 wt. % to about 5 wt. %, from about 0.001 wt. % to about 2 wt. %, from about 0.001 wt. % to about 1 wt. %, from about 0.001 wt. % to about 0.5 wt. %, from about 0.002 wt. % to about 0.5 wt. %, from about 0.005 wt. % to about 0.5 wt. %, from about 0.01 wt. % to about 0.2 wt. %, from about 0.02 wt. % to about 0.03 wt. %, from about 0.03 wt. % to about 0.08 wt. %, from about 0.08 wt. % to about 0.12 wt. %, from about 0.12 wt. % to about 0.16 wt. %, about 0.025 wt. %, about 0.05 wt. %, about 0.10 wt. %, or about 0.15 wt. %.

The compositions described herein are useful at least for preventing a myopathy in poultry when administered to a bird. Besides preventing a myopathy, the compositions described herein may increase the bird's feed intake, increase the bird's weight, improve the quality of breast fillets excised from the bird, increase the bird's feed conversion ratio, or increase the yield of the bird.

### Definitions

Unless otherwise defined herein, scientific and technical terms used in this application shall have the meanings that are commonly understood by those of ordinary skill in the art.

Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art.

Chemistry terms used herein, unless otherwise defined herein, are used according to conventional usage in the art, as exemplified by “The McGraw-Hill Dictionary of Chemical Terms,” Parker S., Ed., McGraw-Hill, San Francisco, C.A. (1985).

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “bird” as used herein refers to members of the superorder Galloanserae.

The term “population of birds” as used herein refers a group of birds comprising at least two birds. In some embodiments, the term population of birds refers to from 2 to 100,000,000 birds, from 2 to 10,000,000 birds, from 2 to 1,000,000 birds, from 2 to 100,000 birds, from 2 to 10,000 birds, from 2 to 1,000 birds, from 2 to 100 birds, from 2 to 10 birds, from 10 to 100,000,000 birds, from 10 to 10,000,000 birds, from 10 to 1,000,000 birds, from 10 to 100,000 birds, from 10 to 10,000 birds, from 10 to 1,000 birds, from 10 to 100 birds, from 100 to 100,000,000 birds, from 100 to 10,000,000 birds, from 100 to 1,000,000 birds, from 100 to 100,000 birds, from 100 to 10,000 birds, from 100 to 1,000 birds, from 1,000 to 100,000,000 birds, from 1,000 to 10,000,000 birds, from 1,000 to 1,000,000 birds, from 1,000 to 100,000 birds, from 1,000 to 10,000 birds, from 10,000 to 100,000,000 birds, from 10,000 to 10,000,000 birds, from 10,000 to 1,000,000 birds, from 10,000 to 100,000 birds, from 100,000 to 100,000,000 birds, from 100,000 to 10,000,000 birds, from 100,000 to 1,000,000 birds, from 1,000,000 to 100,000,000 birds, or from 1,000,000 to 10,000,000 birds.

In some embodiments, evaluation of the efficacy of ASI includes evaluation of a population of birds against another population, each cohort having at least 1,000 birds; in some embodiments, at least 2,000 birds; in some embodiments, at least 3,000 birds; in some embodiments, at least 4,000 birds; in some embodiments, at least 5,000 birds; in some embodiments, at least 10,000 birds; in some embodiments, at least 20,000 birds; and in some embodiments, at least 50,000 birds. In certain embodiments, evaluation of the efficacy of ASI in a population of birds entails an evaluation of a population of a single cohort of at least 1,000 birds; in some embodiments, at least 2,000 birds; in some embodiments, at least 3,000 birds; in some embodiments, at least 4,000 birds; in some embodiments, at least 5,000 birds; in some embodiments, at least 10,000 birds; in some embodiments, at least 20,000 birds; and in some embodiments, at least 50,000 birds, where an efficacious benefit is seen in the population as compared to the evaluated characteristic would have been absent the administration of ASI. In certain embodiments, it is not necessary that the ASI is effective for every individual bird in the population, so long as the population of birds generally experiences a positive effect from the administration of the ASI.

“Treating” a condition, patient, or population of patients refers to taking steps to obtain beneficial or desired results, including clinical results. As used herein, “treatment” is an approach for obtaining beneficial or desired results, including clinical results. Beneficial or desired clinical results can include, but are not limited to, alleviation or amelioration of one or more symptoms or conditions, reduction in severity of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment. Successful treatment may be evaluated, e.g., by evaluating the physical characteristics and/or disease pathology of the subjects or population of subjects to which a composition has been administered relative to a control subject or population of subjects.

The terms “preventing” or “inhibiting,” when used in relation to a condition, such as a local recurrence (e.g., pain), a disease such as myopathy, cancer, a syndrome complex such as heart failure or any other condition, includes administration of a composition which reduces the frequency of, delays the onset of, reduces the severity of, or avoids symptoms of a medical condition in a subject or population of subjects relative to a subject or population of subjects which does not receive the composition. Successful prevention may be evaluated, e.g., by evaluating the physical characteristics and/or disease pathology of the subjects or population of subjects to which a composition has been administered relative to a control subject or population of subjects.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

### EXAMPLES

The invention now being generally described, it will be more readily understood by reference to the following examples, which are included merely for purposes of illustration of certain aspects and embodiments of the present invention, and are not intended to limit the invention.

### Example 1: Adding Arginine Silicate Inositol to Broiler Diet

To test the effect of administering arginine silicate inositol (ASI) to broilers, 1,344 Ross® 708 broilers were divided into 64 pens (21 birds per pen). Each pen was randomly assigned on day 0 to one of four diets. The diets, summarized in Table 1, include a control diet (Diet 1); the control diet supplemented with a blend of arginine, vitamin C, and choline bitartrate (Diet 2); the control diet supplemented with 0.10 wt. % ASI, in which the molar ratio of arginine to silicate to inositol was about 2:2:1 (Diet 3); and the control diet supplemented with 0.002 wt. % astaxanthin (Diet 4). The diets are summarized in Table 1. The broilers were raised on the diets for 49 days.

TABLE 1
Diets Administered to Broilers Diet Key

| | |
|---|---|
| Diet 1 | Control |
| Diet 2 | 0.2022 wt. % arginine + 0.0301 wt. % |
| | vit. C + 0.1737 wt. % choline bitartrate |
| Diet 3 | 0.10 wt. % ASI |
| Diet 4 | 0.002 wt. % astaxanthin |

### Growth Performance Outcomes

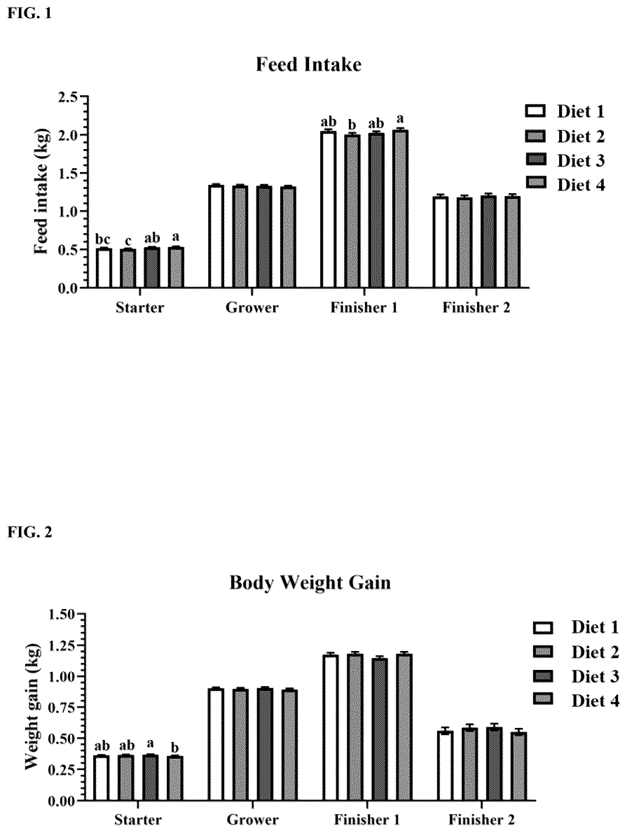

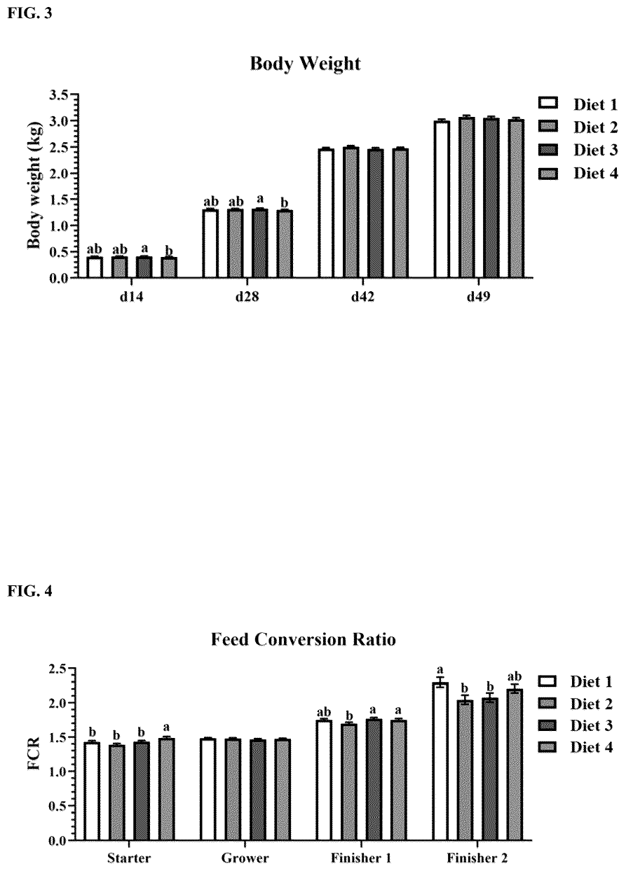

Feed intake, body weight gain, body weight, and feed conversion ratio (FCR) were determined for each pen and averaged by bird at different time points. Data was collected at day 0, the end of the starter period (day 14), the end of the grower period (day 28), the end of the finisher 1 period (day 42), and the end of the finisher 2 period (day 49). Using the data, the feed intake, body weight gain, and FCR were calculated for each period. FCR was calculated by dividing the weight of the feed intake by the weight gain of the bird for each period. The results are summarized in Table 2. The total body weight of the average bird for each diet at the end of each period is also provided in Table 2. Also provided in Table 2 is the standard error of the mean (SEM) and P-value for each set of means. Values with different superscripts within rows indicate means between diets that differed significantly (P≤0.05). The results for feed intake, body weight gain, body weight, and FCR are also depicted in FIG. 1, FIG. 2, FIG. 3, and FIG. 4 respectively.

TABLE 2
Growth Performance Outcomes Averaged by Bird for Each Diet

| Performance | | | | | | Diet |
|---|---|---|---|---|---|---|
| Outcome | Diet 1 | Diet 2 | Diet 3 | Diet 4 | SEM | P-value |
| Feed intake, kg | | | | | | |
| Starter | 0.5197^{bc} | 0.5090^{c} | 0.5292^{ab} | 0.5347^{a} | 0.0053 | 0.0056 |
| Grower | 1.3429 | 1.335 | 1.3327 | 1.3229 | 0.0102 | 0.5546 |
| Finisher 1 | 2.0534^{ab} | 2.0063^{b} | 2.0263^{ab} | 2.0691^{a} | 0.02118 | 0.1693 |
| Finisher 2 | 1.1926 | 1.1805 | 1.2061 | 1.1979 | 0.02489 | 0.8951 |
| Body weight | | | | | | |
| gain, kg | | | | | | |
| Starter | 0.363^{ab} | 0.3667^{ab} | 0.369^{a} | 0.3589^{b} | 0.0031 | 0.1075 |
| Grower | 0.9045 | 0.9004 | 0.9071 | 0.896 | 0.0073 | 0.6904 |
| Finisher 1 | 1.1746 | 1.1818 | 1.1468 | 1.1824 | 0.015 | 0.2954 |
| Finisher 2 | 0.5607 | 0.586 | 0.5909 | 0.551 | 0.0279 | 0.6647 |
| Body weight, kg | | | | | | |
| Day 14 | 0.4066^{ab} | 0.4109^{ab} | 0.4124^{a} | 0.4022^{b} | 0.0032 | 0.0992 |
| Day 28 | 1.3104^{ab} | 1.3128^{ab} | 1.3195^{a} | 1.2954^{b} | 0.0084 | 0.1934 |
| Day 42 | 2.4712 | 2.5048 | 2.4668 | 2.4778 | 0.0188 | 0.4887 |
| Day 49 | 2.9992 | 3.0732 | 3.0529 | 3.0288 | 0.0307 | 0.3495 |
| FCR | | | | | | |
| Starter | 1.4331^{b} | 1.3895^{b} | 1.4345^{b} | 1.4911^{a} | 0.0179 | 0.0024 |
| Grower | 1.4854 | 1.4828 | 1.4697 | 1.4767 | 0.0086 | 0.5432 |
| Finisher 1 | 1.7515a^{b} | 1.6986^{b} | 1.7678^{a} | 1.7531^{a} | 0.0192 | 0.0691 |
| Finisher 2 | 2.2987^{a} | 2.0451^{b} | 2.0753^{b} | 2.2067^{ab} | 0.0719 | 0.0422 |

These results show a trend towards increased growth performance with administration of ASI. Dietary treatment affected feed intake of Ross® 708 broilers in the starter period (P=0.0056), with birds fed Diet 4 eating more than birds fed Diets 1 and 2. Birds fed Diet 3 ate more than birds fed Diet 2 in the first 2 weeks. Birds fed Diet 4 increased feed intake again in the finisher 1 period compared to birds fed Diet 2.

In the starter period, birds fed Diet 3 gained more weight than birds fed Diet 4 as evidenced by the 10.2 g increased day 14 body weight in Diet 3 compared to Diet 4. This increase in body weight continued to the end of the grower period at day 28, where birds fed Diet 3 weighed more than birds fed Diet 4 (24.1 g greater; P=0.0992), which was an unexpectedly superior outcome considering the increased feed intake in birds fed Diet 4.

Feed conversion ratio (FCR) was affected by diet at least in the starter (P=0.0024), finisher 1 (P=0.0691), and finisher 2 (P=0.0422) periods. The increased feed intake without resulting increased weight gain observed in Diet 4 in the starter period increased the starter FCR compared to all other diets. Diet 3 showed a positive impact on broiler body weight at day 14 and day 28, while Diet 2 and Diet 3 both improved FCR in the finisher 2 period.

### Breast Meat Quality

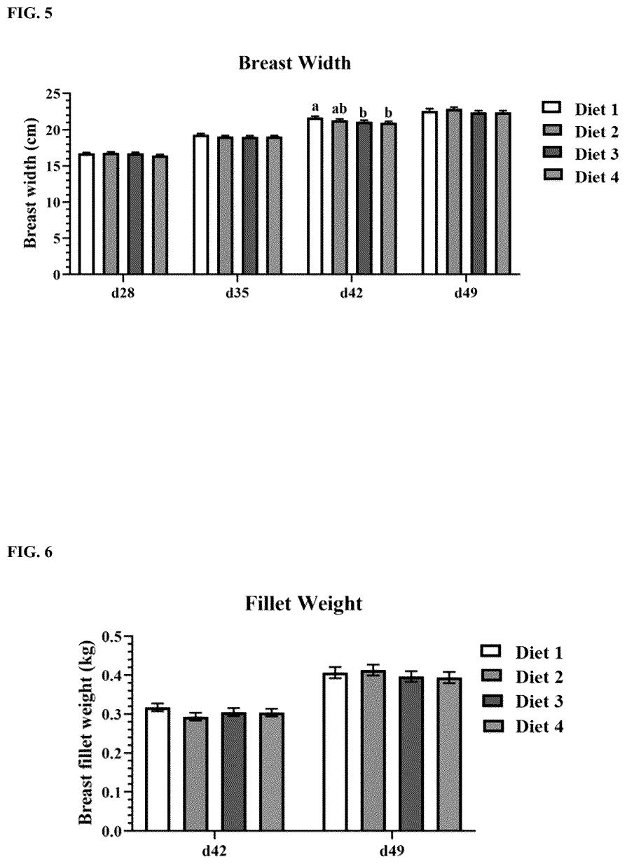

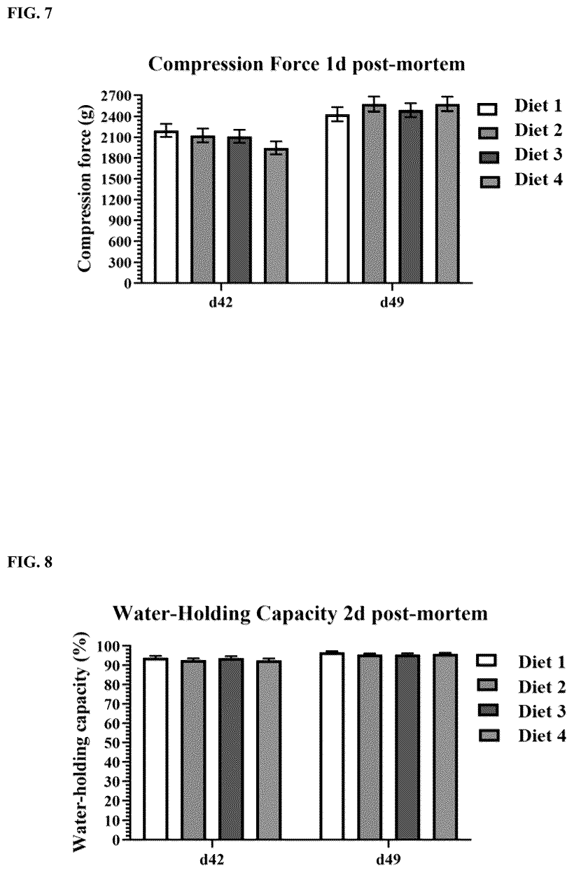

Breast width was measured using seamstress tape measure on 3 birds per pen on day 28, day 35, and day 42, and on 2 birds per pen on day 49. Immediately post-mortem, the left breast fillet was excised from 1 bird per pen on day 42 and day 49 and weighed. Meat quality assays were conducted on the same 64 fillets per time point 1 day (compression force) and 2 days (water-holding capacity) post-mortem. The results are summarized in Table 3. The results for breast width, fillet weight, compression force, and water holding capacity are also depicted in FIG. 5, FIG. 6, FIG. 7, and FIG. 8 respectively.

TABLE 3
Breast Meat Quality Measured on Live Birds and Breast Fillet Post-Mortem

| Breast Quality | | | | | | Diet |
|---|---|---|---|---|---|---|
| Outcome | Diet 1 | Diet 2 | Diet 3 | Diet 4 | SEM | P-value |
| Live bird measure | | | | | | |
| Breast width, cm | | | | | | |
| Day 28 | 16.7348 | 16.8167 | 16.7553 | 16.45 | 0.1353 | 0.2204 |
| Day 35 | 19.3349 | 19.0766 | 19.0489 | 19.083 | 0.1508 | 0.4946 |
| Day 42 | 21.7146^{a} | 21.3435^{ab} | 21.1511^{b} | 21.0106^{b} | 0.1704 | 0.0197 |
| Day 49 | 22.6654 | 22.8767 | 22.43 | 22.4323 | 0.2433 | 0.4448 |
| Breast fillet measure | | | | | | |
| Breast fillet weight, kg | | | | | | |
| Day 42 | 0.3172 | 0.2935 | 0.3054 | 0.3038 | 0.0103 | 0.4254 |
| Day 49 | 0.4064 | 0.4131 | 0.3966 | 0.3938 | 0.01471 | 0.7576 |
| Compression force, g | | | | | | |
| (1 day post-mortem) | | | | | | |
| Day 42 | 2199.4 | 2126.21 | 2113.5 | 1946.65 | 101.1 | 0.2928 |
| Day 49 | 2430.61 | 2574.47 | 2489.14 | 2577.36 | 108.08 | 0.6987 |
| Water holding capacity, % | | | | | | |
| (2 days post-mortem) | | | | | | |
| Day 42 | 93.9719 | 92.75 | 93.8063 | 92.6 | 0.9106 | 0.6158 |
| Day 49 | 96.7067 | 95.5567 | 95.6344 | 95.8813 | 0.5574 | 0.4472 |

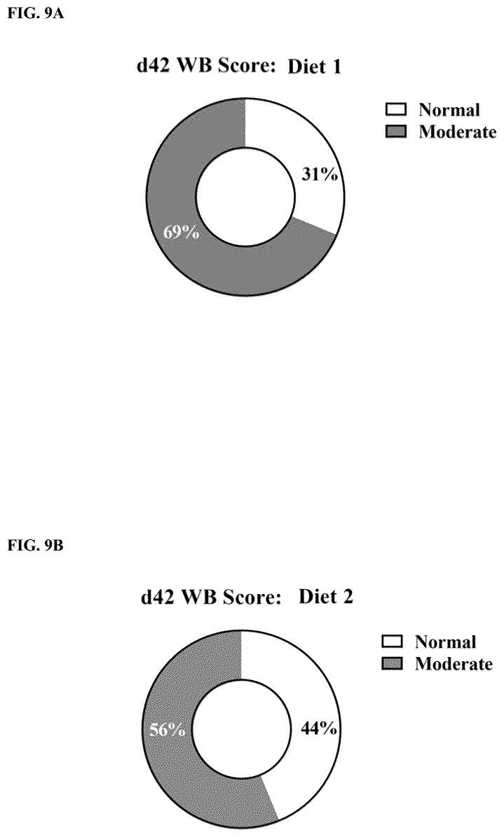

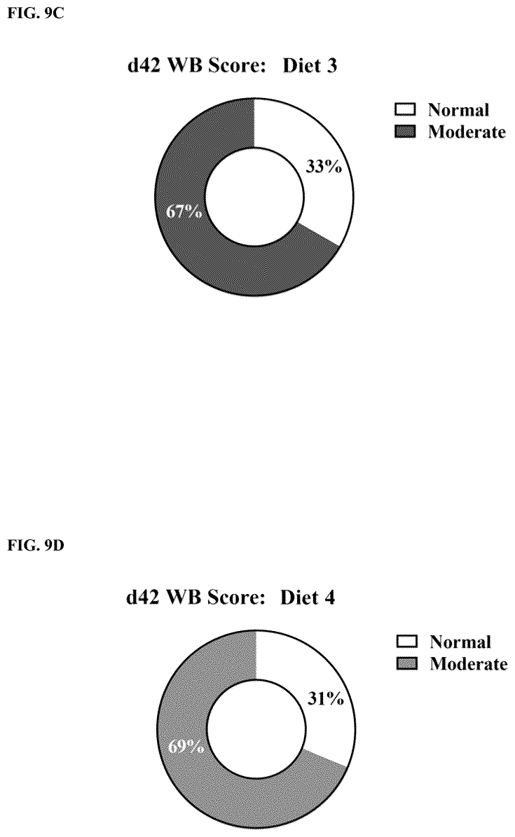

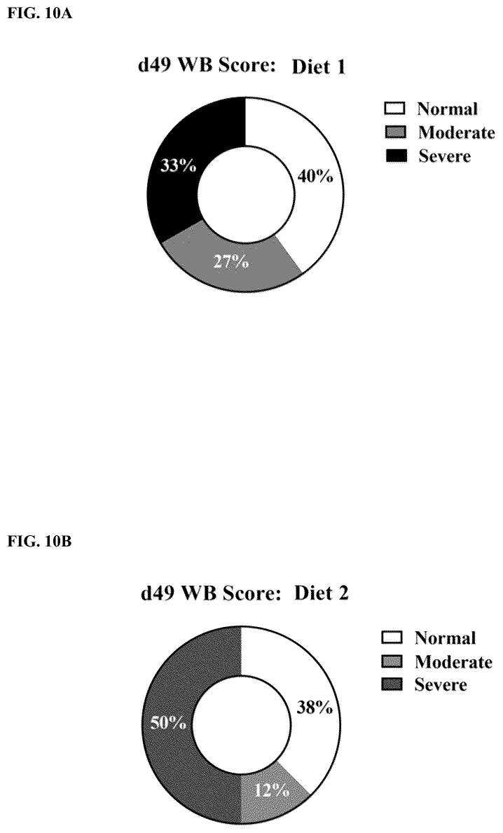

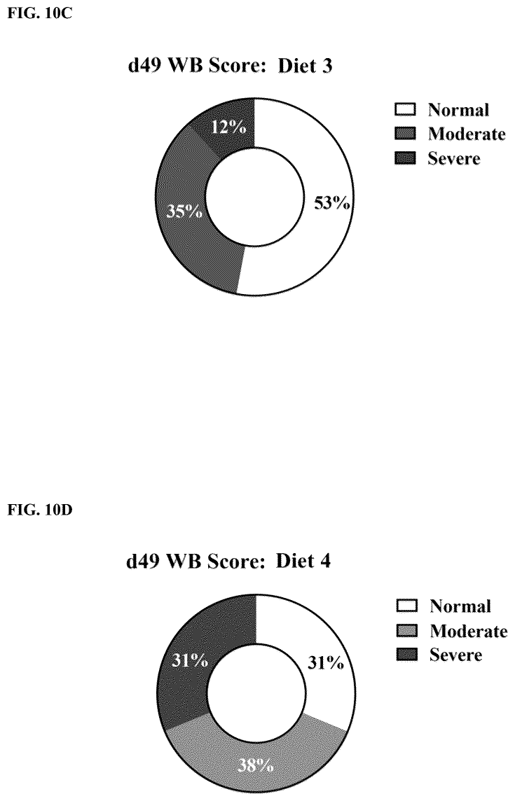

Live birds were palpated for woody breast scores on day 28, day 35, day 42, and day 49, using the same birds that were measured for breast width. Excised breast fillets from 1 bird per pen at day 42 and day 49 were also palpated for woody breast score. Within the live bird scores, there were numerically greater scores of 0 (normal) in Diets 3 and 4 compared to control-fed birds on day 28, day 35, and day 49. When looking at breast fillet scores post-mortem, there were greater scores of 0 (normal) in Diet 2 at day 42 and in Diet 3 at day 49 compared to the control. These preliminary results indicate that Diets 2 and 3 may reduce occurrence of woody breast when the birds are slaughtered after 42 days (Diet 2) and 49 days (Diet 3). The results are summarized in Table 4. For woody breast score distribution analysis, diet P-Value was obtained using the Chi Square Likelihood Ratio analysis due to woody breast score being a categorical variable within the same population, and Likelihood Ratio to determine the ratio of observed vs. expected frequencies. The day 42 and day 49 breast fillet palpation woody breast score results are also depicted in FIGS. 9A-9D and FIGS. 10A-10D respectively.

TABLE 4
Woody Breast Score Percent Distribution by Diet.

| | | | | | |
|---|---|---|---|---|---|
| Woody Breast Score: | | | | | Diet |
| % Distribution by Diet | Diet 1 | Diet 2 | Diet 3 | Diet 4 | P-Value |

Live bird palpation
(Same birds weekly over time)

| | | | | | |
|---|---|---|---|---|---|
| Day 28 | | | | | |
| Score 0: Normal | 73.91 | 70.83 | 80.85 | 85.42 | |
| Score 0.5: Normal-Moderate | 23.91 | 25 | 17.02 | 14.58 | |
| Score 1: Moderate | 2.17 | 4.17 | 2.13 | 0 | 0.4864 |
| Day 35 | | | | | |
| Score 0: Normal | 51.16 | 48.94 | 55.32 | 55.32 | |
| Score 0.5: Normal-Moderate | 0 | 6.38 | 4.26 | 2.13 | |
| Score 1: Moderate | 48.84 | 44.68 | 40.43 | 42.55 | 0.5569 |
| Day 42 | | | | | |
| Score 0: Normal | 53.66 | 43.48 | 53.33 | 55.32 | |
| Score 0.5: Normal-Moderate | 2.44 | 2.17 | 4.44 | 2.13 | |
| Score 1: Moderate | 43.9 | 54.35 | 42.22 | 42.55 | 0.8893 |
| Day 49 | | | | | |
| Score 0: Normal | 7.69 | 23.33 | 36.67 | 32.26 | |
| Score 1: Moderate | 65.38 | 53.33 | 43.33 | 54.84 | |
| Score 2: Severe | 26.92 | 23.33 | 20 | 12.9 | 0.1631 |

Breast fillet palpation
(Selection from birds as above,
but post-mortem)

| | | | | | |
|---|---|---|---|---|---|
| Day 42 | | | | | |
| Score 0: Normal | 31.25 | 43.75 | 26.67 | 31.25 | — |
| Score 0.5: Normal-Moderate | 0 | 0 | 6.67 | 0 | — |
| Score 1: Moderate | 68.75 | 56.25 | 66.67 | 68.75 | 0.6938 |
| Day 49 | | | | | |
| Score 0: Normal | 40 | 37.5 | 52.94 | 31.25 | |
| Score 1: Moderate | 26.67 | 12.5 | 35.29 | 37.5 | |
| Score 2: Severe | 33.33 | 50 | 11.76 | 31.25 | 0.2561 |

### Serum Creatine Kinase

Serum samples from 1 bird per pen (n=64) were submitted to Iowa State University Clinical Pathology laboratory for analysis of creatine kinase (CK) presence. CK is a skeletal muscle-specific enzyme that, when leaked in the serum, is an established indicator of myopathy. Birds fed Diet 3 had lower serum CK than the other diets at day 14, a positive outcome. Diet 3 and Diet 4 had lower serum CK at day 28 than Diet 1 and Diet 2. The results are summarized in Table 5. Data from day 42 and day 49 are missing due to serum creatine kinase levels exceeding the accurate limits of dry chemical analysis performed by the Iowa State University Clinical Pathology Lab.

TABLE 5
Creatine Kinase Presence in Serum of 1 Bird Per Pen

| Creatine Kinase | | | | | | |
|---|---|---|---|---|---|---|
| Presence in Serum | | | | | | Diet |
| Serum CK (iu/L) | Diet 1 | Diet 2 | Diet 3 | Diet 4 | SEM | P-value |
| Day 14 | 2725.53^{ab} | 3198.25^{a} | 1961.14^{b} | 3037.81^{ab} | 400.72 | 0.1293 |
| Day 28 | 7977.69 | 8790.09 | 7025 | 6510.64 | 1029.54 | 0.3682 |
| Day 42 | | | | | | |
| Day 49 | | | | | | |

The reduced serum CK observed in growing broilers fed Diet 3, taken together with the increased body weight at day 14 and day 28, improved FCR in the finisher 2 period, and reduced severity in woody breast scores at day 49 indicate an overall positive effect of this diet on both growth performance and breast muscle quality.

### Example 2: Varying Concentrations of Arginine Silicate Inositol in Broiler Diet

To further study the effect of administering arginine silicate inositol to broilers, Ross® 708 broilers were administered one of five diets at day 0. The diets, summarized in Table 6, include a control diet (Diet 5); the control diet supplemented with 0.025 wt. % of ASI (Diet 6); the control diet supplemented with 0.05 wt. % of ASI (Diet 7); the control diet supplemented with 0.10 wt. % ASI (Diet 8); and the control diet supplemented with 0.15 wt. % ASI (Diet 9). The diets are summarized in Table 6. In all diets containing ASI, the molar ratio of arginine to silicate to inositol was about 2:2:1. The broilers were raised on the diets for 49 days.

TABLE 6
Diets Supplemented with Various Concentrations
of Arginine Silicate Inositol Diet Key

| | |
|---|---|
| Diet 5 | Control |
| Diet 6 | 0.025 wt. % ASI |
| Diet 7 | 0.05 wt. % ASI |
| Diet 8 | 0.10 wt. % ASI |
| Diet 9 | 0.15 wt. % ASI |

### Growth Performance Outcomes

In the same manner as Example 1, feed intake, body weight gain, body weight, and feed conversion ratio (FCR) were determined. The results are summarized in Table 7.

TABLE 7
Growth Performance Outcomes Averaged by Bird for Each Diet

| Performance | | | | | | | Diet |
|---|---|---|---|---|---|---|---|
| Outcome | Diet 5 | Diet 6 | Diet 7 | Diet 8 | Diet 9 | SEM | P-value |
| Feed intake, kg | | | | | | | |
| Starter | 0.410 | 0.412 | 0.409 | 0.419 | 0.423 | 0.010 | 0.842 |
| Grower | 1.677 | 1.668 | 1.650 | 1.647 | 1.652 | 0.014 | 0.441 |
| Finisher 1 | 2.575 | 2.615 | 2.580 | 2.625 | 2.608 | 0.033 | 0.795 |
| Finisher 2 | 1.392 | 1.394 | 1.410 | 1.417 | 1.382 | 0.024 | 0.807 |
| Body weight | | | | | | | |
| gain, kg | | | | | | | |
| Starter | 0.412 | 0.411 | 0.412 | 0.416 | 0.409 | 0.005 | 0.909 |
| Grower | 1.142 | 1.146 | 1.127 | 1.135 | 1.138 | 0.013 | 0.883 |
| Finisher 1 | 1.627 | 1.706 | 1.650 | 1.653 | 1.665 | 0.027 | 0.368 |
| Finisher 2 | 0.623 | 0.586 | 0.625 | 0.627 | 0.606 | 0.025 | 0.797 |
| FCR | | | | | | | |
| Starter | 0.997 | 1.006 | 0.993 | 1.009 | 1.033 | 0.025 | 0.781 |
| Grower | 1.470 | 1.457 | 1.465 | 1.452 | 1.453 | 0.011 | 0.707 |
| Finisher 1 | 1.583^{ab} | 1.535^{b} | 1.564^{ab} | 1.590^{a} | 1.569^{ab} | 0.016 | 0.167 |
| Finisher 2 | 2.277 | 2.397 | 2.294 | 2.288 | 2.295 | 0.075 | 0.834 |
| Body weight (kg) | | | | | | | |
| Day 14 | 0.456 | 0.455 | 0.456 | 0.460 | 0.454 | 0.005 | 0.920 |
| Day 28 | 1.598 | 1.596 | 1.583 | 1.596 | 1.588 | 0.014 | 0.914 |
| Day 42 | 3.221 | 3.310 | 3.242 | 3.257 | 3.273 | 0.030 | 0.324 |
| Day 49 | 3.797 | 3.835 | 3.866 | 3.884 | 3.878 | 0.035 | 0.421 |

In a pairwise comparison between all diets, feed conversion ratio was improved in the Finisher 1 period in Diet 6 compared to the Diet 8 by a 5-point decrease, indicating an advantage during this performance period to Diet 6. Numerically, it is worth noting that birds fed Diet 6 showed the greatest body weight at day 42 (3.310 kg) while birds fed Diet 8 finished with the greatest average body weight of all diets at day 49 (3.884 kg). Additionally, it appears that up to 0.15% ASI does not affect feed palatability or provide a significantly negative impact on any performance measure.

### Breast Meat Quality

Breast width was measured using seamstress tape measure on 1 bird per pen on day 42, and day 49. Numerically, broilers fed Diet 6 had the greatest measured breast width on day 49, averaging 0.5 cm greater than birds fed Diet 8, although the Diet 8-fed broilers had the numerically greatest body weight at this time point. Immediately post-mortem, the left breast fillet was excised from the same bird on day 42 and day 49 and weighed. Meat quality assays were conducted the same fillets per timepoint 1 day (compression force) and 2 days (water-holding capacity) post-mortem. Interestingly, breasts from birds fed both Diet 6 and Diet 8 showed numerically decreased compression force, indicative of greater meat tenderness, at day 42. Diet 6 breasts averaged 199 g of force less than the control, and Diet 8 breasts averaged 271 g of force less than the control at this time point. This effect continued through day 49, with Diet 6 breasts averaging 308 g of force less than the control, and Diet 8 breasts averaging 349 g of force less than the control. Diet 7 and Diet 9 also showed numerically decreased compression force compared to the control at day 49, indicating an overall improvement from all ASI-containing diets on this outcome at 49 days. The results are summarized in Table 8.

TABLE 8
Breast Meat Quality Measured on Live Birds and Breast Fillet Post-Mortem

| Breast Quality | | | | | | | Diet |
|---|---|---|---|---|---|---|---|
| Outcome | Diet 5 | Diet 6 | Diet 7 | Diet 8 | Diet 9 | SEM | P-value |
| Live bird measure | | | | | | | |
| Breast width, cm | | | | | | | |
| Day 42 | 25.75 | 25.17 | 25.92 | 25.83 | 25.67 | 0.558 | 0.890 |
| Day 49 | 27.38 | 28.17 | 28 | 27.67 | 27.46 | 0.482 | 0.733 |
| Breast fillet | | | | | | | |
| measure | | | | | | | |
| Breast fillet | | | | | | | |
| weight, kg | | | | | | | |
| Day 42 | 0.408 | 0.413 | 0.435 | 0.428 | 0.433 | 0.018 | 0.768 |
| Day 49 | 0.501 | 0.507 | 0.530 | 0.494 | 0.512 | 0.022 | 0.821 |
| Compression | | | | | | | |
| force, g (1 day | | | | | | | |
| post-mortem) | | | | | | | |
| Day 42 | 3092.42 | 2893.51 | 3231.76 | 2821.22 | 3229 | 164.78 | 0.251 |
| Day 49 | 4033.19 | 3725.76 | 3554.33 | 3684.44 | 3480.46 | 273.34 | 0.650 |
| Water holding | | | | | | | |
| capacity, % (2 | | | | | | | |
| days post-mortem) | | | | | | | |
| Day 42 | 96.19 | 95.9 | 95.91 | 96.87 | 96.57 | 0.633 | 0.750 |
| Day 49 | 93.85 | 95.56 | 93.76 | 94.87 | 94.58 | 0.927 | 0.597 |

Excised breast fillets from 1 bird per pen at day 42 and day 49 were palpated for woody breast score and visually scored for white striping using a normal (0), moderate (1), and severe (3) scale for each outcome. The distribution of normal woody breast scores on day 42 was increased in Diet 6, Diet 7, and Diet 9 compared to the control, with the largest proportion of normal breasts observed in Diet 6 (66.67% of scores compared to only 25% of control scores). Diet 6 and Diet 7 had the lowest percent of severe scores at day 42 (8.33% in both diets) compared to the control (16.67% of scores were severe). At day 49, a similar shift in woody breast scores was observed, with all ASI diets having increased normal scores compared to the control. Diets 8 and 9 showed the greatest percentage of normal breasts (33.33%), while Diets 7 and 8 showed the lowest percentage of severe scores: 25% each compared to 58.33% of control breasts scored severe at this time point. ASI inclusion in the diet appeared to have an overall, observable effect on diminishing woody breast severity at slaughter age. The woody breast results are summarized in Table 9. For woody breast score distribution analysis, diet P-Value was obtained using the Chi Square Likelihood Ratio analysis due to woody breast score being a categorical variable within the same population, and Likelihood Ratio to determine the ratio of observed vs. expected frequencies.

TABLE 9
Woody Breast Score Percent Distribution by Diet

| Woody Breast Score: | | | | | | Diet |
|---|---|---|---|---|---|---|
| % Distribution by Diet | Diet 5 | Diet 6 | Diet 7 | Diet 8 | Diet 9 | P-Value |
| Day 42 | | | | | | |
| Score 0: Normal | 25 | 66.67 | 25 | 58.33 | 41.67 | 0.195 |
| Score 1: Moderate | 58.33 | 25 | 66.67 | 16.67 | 41.67 | |
| Score 2: Severe | 16.67 | 8.33 | 8.33 | 25 | 16.67 | |
| Day 49 | | | | | | |
| Score 0: Normal | 0 | 16.67 | 8.33 | 33.33 | 33.33 | 0.112 |
| Score 1: Moderate | 41.67 | 33.33 | 66.67 | 41.67 | 25 | |
| Score 2: Severe | 58.33 | 50 | 25 | 25 | 41.67 | |

Regarding white striping, Diet 6 breasts had the greatest percentage of normal scores at day 42 (66.67% compared to 58.33% in the control). At day 49, all ASI diet breasts showed improved white striping scores compared to the control diet, with the greatest percentage of normal scores (50%) observed in Diets 6, 8, and 9. Notably, no Diet 6 breasts scored severe at day 49. The white striping score results are summarized in Table 10. For white striping score distribution analysis, diet P-Value was obtained using the Chi Square Likelihood Ratio analysis due to white striping score being a categorical variable within the same population, and Likelihood Ratio to determine the ratio of observed vs. expected frequencies.

TABLE 10
White Striping Score Percent Distribution by Diet

| White Striping Score: | | | | | | Diet |
|---|---|---|---|---|---|---|
| % Distribution by Diet | Diet 5 | Diet 6 | Diet 7 | Diet 8 | Diet 9 | P-Value |
| Day 42 | | | | | | |
| Score 0: Normal | 58.33 | 66.67 | 58.33 | 50 | 58.33 | 0.807 |
| Score 1: Moderate | 41.67 | 25 | 33.33 | 50 | 33.33 | |
| Score 2: Severe | 0 | 8.33 | 8.33 | 0 | 8.33 | |
| Day 49 | | | | | | |
| Score 0: Normal | 16.67 | 50 | 33.33 | 50 | 50 | 0.324 |
| Score 1: Moderate | 58.33 | 50 | 41.67 | 33.33 | 33.33 | |
| Score 2: Severe | 25 | 0 | 25 | 16.67 | 16.67 | |

### Serum Creatine Kinase and Myoglobin

Serum samples from 1 bird per pen (n=60) from day 14, day 28, day 42, and day 49 were analyzed for presence of creatine kinase (CK) and myoglobin using competitive ELISA assays. CK is a skeletal muscle-specific enzyme that, when leaked in the serum, is an established indicator of myopathy. Myoglobin is a protein that serves to bind oxygen in muscle cells and plays the defining role in meat color. Similar to CK, myoglobin released into circulation shows muscle damage. In both cases, reduction in the serum would indicate a protective effect on muscle. Numerically, Diet 8 alone decreased serum CK on day 49 compared to the control (1.59 ng/Ul decrease). In a pairwise comparison between all diets, serum myoglobin was decreased in the serum of birds fed Diet 6 on day 42 compared to the control (8.72 ng/Ul decrease, P<0.05). The CK results are summarized in Table 11 and the Myoglobin results are summarized in Table 12.

TABLE 11
Creatine Kinase Measured in the Serum of 1 Bird Per Pen

| Creatine Kinase | | | | | | | |
|---|---|---|---|---|---|---|---|
| Presence in Serum | | | | | | | Diet |
| Serum CK (ng/Ul) | Diet 5 | Diet 6 | Diet 7 | Diet 8 | Diet 9 | SEM | P-value |
| Day 14 | 48.36 | 48.41 | 49.34 | 48.70 | 49.43 | 0.0859 | 0.838 |
| Day 28 | 48.56 | 48.61 | 48.79 | 49.18 | 48.85 | 0.723 | 0.975 |
| Day 42 | 54.30 | 51.85 | 53.80 | 52.29 | 53.37 | 1.33 | 0.593 |
| Day 49 | 77.87 | 84.51 | 83 | 76.28 | 77.05 | 4.50 | 0.560 |

TABLE 12
Myoglobin Measured in the Serum of 1 Bird Per Pen

| Serum Myoglobin | | | | | | | |
|---|---|---|---|---|---|---|---|
| Concentration | | | | | | | |
| Serum myoglobin | | | | | | | Diet |
| (ng/Ul) | Diet 5 | Diet 6 | Diet 7 | Diet 8 | Diet 9 | SEM | P-value |
| Day 14 | 175.36 | 179.62 | 178.75 | 178.47 | 180.4 | 3.575 | 0.856 |
| Day 28 | 186.67 | 186.77 | 188.84 | 185.44 | 186.38 | 3.319 | 0.946 |
| Day 42 | 200.12^{a} | 191.4^{b} | 196^{ab} | 196.21^{ab} | 195.69^{ab} | 2.941 | 0.269 |
| Day 49 | 188.69 | 188.09 | 187.13 | 189.11 | 186.39 | 3.169 | 0.972 |

There appears to be an overall benefit of ASI dietary inclusion on woody breast severity in broilers with no negative impact on growth performance. Diet 6 (0.025 wt. % ASI) and Diet 8 (0.10 wt. % ASI) showed the greatest benefits. Birds fed Diet 6 showed improved FCR in the finisher 1 period with the greatest numerical breast width on day 49, as well as numerically decreased compression force on day 42 and day 49. Furthermore, Diet 6 breasts had the greatest percentage of both normal woody breast and white striping scores at day 42 and showed 0% severe white striping at day 49. Birds fed Diet 6 exhibited decreased serum myoglobin compared to the control on day 42, the only diet to show this protective effect.

Birds fed Diet 8 had the numerically greatest body weights at day 49. Diet 8 breasts also showed numerically decreased compression force compared to the control at day 42 and day 49, and reduced severity of woody breast and white striping scores at day 42 and day 49. Diet 8 breasts had the lowest percentage of severe woody breast scores at day 49 and had no severe white striping scores at day 42. Additionally, birds fed Diet 8 showed a numerical decrease in serum CK at day 49.

### INCORPORATION BY REFERENCE

All publications and patents mentioned herein are hereby incorporated by reference in their entirety. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

We claim:

1. A method of feeding a bird, comprising feeding a composition comprising arginine silicate inositol to the bird.

2. A method of feeding a population of birds, comprising feeding a composition comprising arginine silicate inositol to the population of birds.

3. The method of claim 1 or claim 2, wherein the method is a method for treating or preventing a myopathy in the bird or population of birds.

4. A method for treating, inhibiting, or preventing a myopathy comprising administering arginine silicate inositol to a bird.

5. The method of claim 3 or claim 4, wherein the method reduces the severity of the myopathy.

6. The method of any one of claims 3-5, wherein the myopathy is white striping or woody breast.

7. The method of any one of claims 3-6, wherein the myopathy is woody breast.

8. A method for improving poultry breast fillet quality comprising administering arginine silicate inositol to a bird.

9. A method for improving poultry breast fillet quality comprising administering arginine silicate inositol to a population of birds.

10. The method of claim 9, wherein the breast fillet quality is improved relative to a population of birds to which the arginine silicate inositol is not administered.

11. A method for increasing poultry weight comprising administering arginine silicate inositol to a bird.

12. A method for increasing poultry weight comprising administering arginine silicate inositol to a population of birds.

13. The method of claim 12, wherein the average weight of the population of birds is increased relative to a population of birds to which the arginine silicate inositol is not administered.

14. The method of any one of claims 1-13, wherein the bird is a chicken.

15. The method of claim 14, wherein the chicken is a broiler.

16. The method of any one of claims 1-15, wherein the arginine silicate inositol or composition comprising arginine silicate inositol is administered orally.

17. The method of any one of claims 1-15, wherein the arginine silicate inositol or composition comprising arginine silicate inositol is administered parenterally.

18. The method of any one of claims 1-15, wherein the arginine silicate inositol or composition comprising arginine silicate inositol is administered intramuscularly.

19. The method of any one of claims 1-15, wherein the arginine silicate inositol or composition comprising arginine silicate inositol is administered intranasally.

20. The method of any one of claims 1-15, wherein the arginine silicate inositol or composition comprising arginine silicate inositol is administered under a wing of the bird.

21. The method of any one of claims 1-20, wherein the arginine silicate inositol is administered as a composition comprising arginine silicate inositol and at least one carrier, or wherein the composition comprising arginine silicate inositol further comprises at least one carrier.

22. The method of claim 21, wherein the composition is a liquid composition.

23. The method of claim 22, wherein the carrier is water or an oil.

24. The method of claim 22 or claim 23, wherein the liquid composition is an emulsion, a suspension, or a solution.

25. The method of any one of claims 21-23, wherein the liquid composition further comprises a sweetener, a flavoring agent, a coloring agent, a preservative, a solubilizer, a wetting agent, a stabilizer, an antioxidant, a diluent, or a combination thereof.

26. The method of claim 21, wherein the composition is a solid composition.

27. The method of claim 26, wherein the carrier is an animal feed.

28. The method of claim 27, wherein the animal feed comprises a cereal grain, preferably wherein the animal feed comprises from about 55 wt % to about 80 wt % cereal grain.

29. The method of claim 27 or claim 28, wherein the animal feed comprises an oilseed meal, preferably wherein the animal feed comprises from about 15 wt. % to about 25 wt. % oilseed meal.

30. The method of any one of claims 27-29, wherein the animal feed comprises from about 12 wt. % to about 30 wt. % protein.

31. The method of any one of claims 27-30, wherein the animal feed comprises a cereal grain selected from corn, soybean, wheat, or a combination thereof.

32. The method of any one of claims 27-31, wherein the animal feed comprises a coccidiostat, an antibiotic, or a combination thereof.

33. The method of any one of claims 27-32, wherein the animal feed comprises an added digestible amino acid other than arginine.

34. The method of any one of claims 26-33, wherein the solid composition is formulated as a mash.

35. The method of any one of claims 26-33, wherein the solid composition is formulated as a crumble.

36. The method of any one of claims 26-33, wherein the solid composition is formulated as pellets.

37. The method of any one of claims 26-33, wherein the solid composition is formulated as a scratch grain.

38. The method of any one of claims 26-37, wherein the solid composition is adapted for a starter period.

39. The method of claim 38, wherein the solid composition is adapted to provide a feed conversion ratio of from about 0.8 to about 1.0 during the starter period.

40. The method of any one of claims 26-37, wherein the solid composition is adapted for a grower period.

41. The method of claim 40, wherein the solid composition is adapted to provide a feed conversion ratio of from about 1.0 to about 1.47 during the grower period.

42. The method of any one of claims 26-37, wherein the solid composition is adapted for a finisher 1 period.

43. The method of claim 42, wherein the solid composition is adapted to provide a feed conversion ratio of from about 1.4 to about 1.55 during the finisher 1 period.

44. The method of any one of claims 26-37, wherein the solid composition is adapted for a finisher 2 period.

45. The method of claim 44, wherein the solid composition is adapted to provide a feed conversion ratio of from about 2.2 to about 2.3 during the finisher 2 period.

46. The method of any one of claims 26-45, wherein the bird is administered from 0.1 kg to 10 kg of the solid composition.

47. The method of any one of claims 21-46, wherein the composition comprises from about 0.001 wt. % to about 0.5 wt. % of the arginine silicate inositol.

48. The method of claim 47, wherein the composition comprises about 0.025 wt %, about 0.05 wt %, about 0.10 wt %, or about 0.15 wt % of the arginine silicate inositol.

49. The method of any one of claims 1-48, wherein the arginine silicate inositol is administered over from 1 day to 55 days.

50. The method of any one of claims 1-48, wherein the arginine silicate inositol is administered over from 5 days to 42 days.

51. The method of any one of claims 1-48, wherein the arginine silicate inositol is administered over from 7 days to 14 days.

52. The method of any one of claims 1-48, wherein the method comprises slaughtering the bird at an age of from 38 days to 55 days.

53. The method of claim 52, wherein the bird weighs from about 2.4 kg to about 4.00 kg when slaughtered.

54. A solid composition comprising from about 0.005 wt. % to about 0.5 wt. % arginine silicate inositol and at least one carrier.

55. The composition of claim 54, wherein the carrier is an animal feed.

56. The composition of claim 55, wherein the animal feed comprises a cereal grain, preferably wherein the animal feed comprises from about 55 wt % to about 80 wt % cereal grain.

57. The composition of claim 55 or claim 56, wherein the animal feed comprises an oilseed meal, preferably wherein the feed comprises from about 15 wt. % to about 25 wt. % oilseed meal.

58. The composition of any one of claims 55-57, wherein the animal feed comprises from about 12 wt. % to about 30 wt. % protein.

59. The composition of any one of claims 55-58, wherein the animal feed comprises a cereal grain selected from corn, soybean, wheat, or a combination thereof.

60. The composition of any one of claims 55-59, wherein the animal feed comprises a coccidiostat, an antibiotic, or a combination thereof.

61. The composition of any one of claims 55-60, wherein the animal feed comprises an added digestible amino acid other than arginine.

62. The composition of any one of claims 54-61, formulated as a mash.

63. The composition of any one of claims 54-61, formulated as a crumble.

64. The composition of any one of claims 54-61, formulated as pellets.

65. The composition of any one of claims 54-61, formulated as a scratch grain.

66. The composition of any one of claims 54-65, adapted for a starter period.

67. The composition of claim 66, adapted to provide a feed conversion ratio of from about 0.8 to about 1.0 during the starter period.

68. The composition of any one of claims 54-65, adapted for a grower period.

69. The composition of claim 68, adapted to provide a feed conversion ratio of from about 1.0 to about 1.47 during the grower period.

70. The composition of any one of claims 54-65, adapted for a finisher 1 period.

71. The composition of claim 70, adapted to provide a feed conversion ratio of from about 1.4 to about 1.55 during the finisher 1 period.

72. The composition of any one of claims 54-65, adapted for a finisher 2 period.

73. The composition of claim 72, adapted to provide a feed conversion ratio of from about 2.2 to about 2.3 during the finisher 2 period.
